(19) United States
(12) Patent Application Publication
(10) Pub. No.: US 2024/0100220 A1
(43) Pub. Date: Mar. 28, 2024

(54) BIOACTIVE IPMLANTS AND METHODS OF MAKING

(71) Applicant: Advanced Biologics, LLC, Carlsbad, CA (US)

(72) Inventors: Amit Prakash GOVIL, Carlsbad, CA (US); Bryan John CHOI, Carlsbad, CA (US); Vanitra Antoinette RICHARDSON, Carlsbad, CA (US)

(21) Appl. No.: 18/261,892

(22) PCT Filed: Jan. 18, 2022
(86) PCT No.: PCT/US2022/012766

§ 371 Date: Jul. 18, 2023

Related U.S. Application Data
(60) Provisional application No. 63/138,683, filed on Jan. 18, 2021.

(51) Int. Cl. A61L 27/22; A61L 27/04; A61L 27/36

(52) U.S. Cl. CPC A61L 27/227; A61L 27/047; A61L 27/3608; A61L 27/365; A61L 2300/414; A61L 2430/02

(57) ABSTRACT

Described herein are methods to increase growth factor binding when demineralizing bone. Also described herein are methods of enriching bound growth factors in a medical implant.

### CROSS-REFERENCE TO RELATED APPLICATIONS

This application claims benefit of U.S. Provisional Application No. 63/138,683, filed Jan. 18, 2021, which is hereby incorporated herein by reference in its entirety.

### BACKGROUND

Correction of musculoskeletal tissue defects in humans is a significant problem that faces the field of medicine, especially that of orthopedic surgery. Currently available implants, which aim to correct such defects, have limited capacity to interact with the environment in which they are implanted. This limited capacity to interact with the host tissue precludes such implants from reaching their full potential, and in many cases, can prevent successful outcomes for such implants.

### SUMMARY

Described herein are methods to increase growth factor binding during bone demineralization. Methods of increasing growth factor binding as described herein can comprise providing a bone composition; adding an acid to the bone composition, thereby exposing the bone composition to an acid to demineralize the bone composition; optionally repeating this step by decanting the acid solution and adding a new acid solution until a target pH is reached suitable for demineralization; and adding a buffer or base to the acidic solution in an amount effective to precipitate minerals and promote binding of soluble growth factors in the acid solution to the bone composition, precipitated minerals, or a combination thereof. Methods to increase growth factor binding can also comprise isolating the bone composition, mineral that has precipitated of solution, or both. In certain aspects, the extracellular matrix of the bone and/or bone composition can be removed prior to adding an acid to the solution.

Also described herein are methods of enriching bound growth factors in a medical implant. Such methods comprise providing a scaffold; combining the scaffold with growth factors or agents; and raising the pH of the solution from a first level to a second level, wherein the second level is more basic than the first, by adding a base or buffer to the solution. Such methods can further comprise isolating the scaffold. The scaffold can be derived from allograft or xenograft tissue sources or created synthetically. The scaffold can be comprised of comprised of collagen, hyaluronan, or other protein, amino acid, GAG, and/or biocompatible material.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a flow chart illustrating an embodiment of the method 1000.

FIG. 2 is a flow chart illustrating an embodiment of the method 2000.

FIG. 3 is a flow chart illustrating an embodiment of the method 3000.

FIG. 4 is a flow chart illustrating an embodiment of the method 4000.

FIG. 5 is a flow chart illustrating an embodiment of the method 5000.

FIGS. 6A to 6B are bar graphs showing effect of pH after neutralization on aFGF (FIG. 6A), bFGF (FIG. 6B), and VEGF (FIG. 6C).

FIG. 7 illustrates a study protocol.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, physiology, modern surgical techniques, microbiology, organic chemistry, biochemistry, and the like, which are within the skill of the art. Such techniques are explained fully in the literature.

In describing the disclosed subject matter, the following terminology will be used in accordance with the definitions set forth below.

As used herein, “about,” “approximately,” and the like, when used in connection with a numerical variable, generally refers to the value of the variable and to all values of the variable that are within the experimental error (e.g., within the 95% confidence interval for the mean) or within +−0.10% of the indicated value, whichever is greater.

As used herein, ““effective amount” is an amount sufficient to effect beneficial or desired results. An effective amount can be administered in one or more administrations, applications, or dosages.

As used herein, “therapeutic” refers to treating or curing a disease or condition.

As used herein, “preventative” refers to hindering or stopping a disease or condition before it occurs or while the disease or condition is still in the sub-clinical phase.

As used herein, “concentrated” used in reference to an amount of a molecule, compound, or composition, including, but not limited to, a chemical compound, polynucleotide, peptide, polypeptide, protein, antibody, or fragments thereof, that indicates that the sample is distinguishable from its naturally occurring counterpart in that the concentration or number of molecules per volume is greater than that of its naturally occurring counterpart.

As used herein, “isolated” means separated from constituents, cellular and otherwise, with which the polynucleotide, peptide, polypeptide, protein, antibody, or fragments thereof, are normally associated in nature. A non-naturally occurring polynucleotide, peptide, polypeptide, protein, antibody, or fragments thereof, does not require “isolation” to distinguish it from its naturally occurring counterpart.

As used herein, “diluted” used in reference to an amount of a molecule, compound, or composition including but not limited to, a chemical compound, polynucleotide, peptide, polypeptide, protein, antibody, or fragments thereof, that indicates that the sample is distinguishable from its naturally occurring counterpart in that the concentration or number of molecules per volume is less than that of its naturally occurring counterpart.

As used interchangeably herein, “subject,” “individual,” or “patient,” refers to a vertebrate, preferably a mammal, more preferably a human. Mammals include, but are not limited to, murines, simians, humans, farm animals, sport animals, and pets. The term “pet” includes a dog, cat, guinea pig, mouse, rat, rabbit, ferret, and the like. The term farm animal includes a horse, sheep, goat, chicken, pig, cow, donkey, llama, alpaca, turkey, and the like.

As used herein, “biocompatible” or “biocompatibility” refers to the ability of a material to be used by a patient without eliciting an adverse or otherwise inappropriate host response in the patient to the material or a derivative thereof, such as a metabolite, as compared to the host response in a normal or control patient.

As used herein, “cell,” “cell line,” and “cell culture” include progeny. It is also understood that all progeny may not be precisely identical in DNA content, due to deliberate or inadvertent mutations. Variant progeny that have the same function or biological property, as screened for in the originally transformed cell, are included.

As used herein, “specific binding” refers to binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody preferably binds to a single epitope and to no other epitope within the family of proteins.

As used herein, “control” is an alternative subject or sample used in an experiment for comparison purposes and included to minimize or distinguish the effect of variables other than an independent variable. As used herein, “positive control” refers to a “control” that is designed to produce the desired result, provided that all reagents are functioning properly and that the experiment is properly conducted.

As used herein, “negative control” refers to a “control” that is designed to produce no effect or result, provided that all reagents are functioning properly and that the experiment is properly conducted. Other terms that are interchangeable with “negative control” include “sham,” “placebo,” and “mock.”

As used herein, “culturing” refers to maintaining cells under conditions in which they can proliferate and avoid senescence as a group of cells. “Culturing” can also include conditions in which the cells also or alternatively differentiate.

As used herein, “synergistic effect,” “synergism,” or “synergy” refers to an effect arising between two or more molecules, compounds, substances, factors, or compositions that is greater than or different from the sum of their individual effects.

As used herein, “additive effect” refers to an effect arising between two or more molecules, compounds, substances, factors, or compositions that is equal to or the same as the sum of their individual effects.

As used herein, “autologous” refers to being derived from the same subject that is the recipient.

As used herein, “allograft” refers to a graft that is derived from one member of a species and grafted in a genetically dissimilar member of the same species.

As used herein “xenograft” or “xenogeneic” refers to a substance or graft that is derived from one member of a species and grafted or used in a member of a different species.

As used herein, “autograft” refers to a graft that is derived from a subject and grafted into the same subject from which the graft was derived.

As used herein, “allogeneic” refers to involving, derived from, or being individuals of the same species that are sufficiently genetically different so as to interact with one another antigenicaly.

As used herein, “syngeneic” refers to subjects or donors that are genetically similar enough so as to be immunologically compatible to allow for transplantation, grafting, or implantation.

As used herein, “implant” or “graft,” as used interchangeably herein, refers to cells, tissues, or other compounds, including metals and plastics, that are inserted into the body of a subject.

As use herein, “immunogenic” or “immunogenicity” refers to the ability of a substance, compound, molecule, and the like (referred to as an “antigen”) to provoke an immune response in a subject.

As used herein, “exogenous” refers to a compound, substance, or molecule coming from outside a subject or donor, including their cells and tissues.

As used herein, “endogenous” refers to a compound, substance, or molecule originating from within a subject or donor, including their cells or tissues.

As used herein, “bioactive” refers to the ability or characteristic of a material, compound, molecule, or other particle that interacts with or causes an effect on any cell, tissue and/or other biological pathway in a subject.

As used herein, “bioactive factor” refers to a compound, molecule, or other particle that interacts with or causes an effect on any cell, tissue, and/or other biological pathway in a subject.

As used herein, “physiological solution” refers to a solution that is about isotonic with tissue fluids, blood, or cells.

As used herein, “donor” refers to a subject from which cells or tissues are derived.

As used herein, “slurry” refers to the resultant product from any of the methods described herein. Accordingly, the slurry can be in any form resulting from the processing described herein, including but not limited to, dehydrated slurry or tissue, paste, powder, solution, gel, putty, particulate and the like.

As used herein, “extra cellular matrix” refers to the non-cellular component surrounding cells that provides support functions to the cell including structural, biochemical, and biophysical support, including but not limited to, providing nutrients, scaffolding for structural support, and sending or responding to biological cues for cellular processes such as growth, differentiation, and homeostasis.

As used herein, “complete extracellular matrix” refers to extracellular matrix that has all components (proteins, peptides, proteoglycans, and the like) present and may or may not include other cells that are embedded in the extra cellular matrix.

As used herein, “decellularized extracellular matrix” refers to complete extracellular matrix that has been processed to remove any cells embedded within the extracellular matrix.

As used herein, “extracellular matrix component” refers to a particular component. By way of a non-limiting example, an extracellular matrix comportment can be a specific class of comments (e.g., proteoglycans) or individual component (e.g., collagen I) that is separated or isolated from the other extracellular components. These components can be made synthetically. As used herein “hydrogel” refers to a network of hydrophilic polymer chains that are dispersed in water. “Hydrogel” also includes a network of hydrophilic polymer chains dispersed in water that are found as a colloidal gel.

As used herein, “adipocyte” refers to a cell type also known as a lipocyte or fat cell. Adipocytes are the cells that primarily compose adipose tissue, specialized in storing energy as fat.

As used herein, “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

As used herein, “effective amount” refers to an effective amount of medical implants as described herein to reduce the appearance of subcutaneous soft-tissue defects, to bolster the support of endogenous soft-tissue in the body which supports joints (for example, ankle joints, knee joints, vertebral discs, and the like, or to aid in the cushioning provided by soft-tissue at joints or on pads of the hands and/or feet.

As used herein, “primarily” means that a part of the whole is present in an amount greater than all other parts of the whole, greater than all other parts individually or in combination.

Described herein are bioactive implants (also referred to herein as medical implants), methods of making, methods of delivery, and kits comprising bioactive implants.

As described herein, medical implants can comprise collagen, hyaluronan, protein, amino acid, peptide, and glycosaminoglycan. In embodiments of the present disclosure, the medical implants are primarily collagen-based and derived from allograft tissue. In embodiments according to the present disclosure, proteins and peptides are proteins and peptides derived from allograft tissue.

In certain aspects, medical implants can further comprise biocompatible material. In certain aspects, biocompatible material can be material that can product medical implants during processing and/or storage. In embodiments of the present disclosure, the biocompatible material is one or more of glycerol, propylene glycol, sugars, and preservatives, individually or in combination.

In certain aspects, medical implants can further comprise growth factors.

In certain aspects, medical implants can comprise a plurality of cells from one or more sources.

The cell or growth factors of the present disclosure can each independently be derived from adipose, bone, periosteum, endosteum, cartilage, synovial fluid, tendon, ligament, skin, adipose, fascia, bone marrow, blood, muscle, amniotic fluid, or from another matrix such as intestine, bladder, placental, exosomes, or other soft tissue. Sources of such can be allograft, autograft, xenograft, or synthetic sources.

In certain embodiments, medical implants are acellular.

In certain aspects, medical implants can be delipidized. Lipids can be removed from medical implants according to well-known methods as known by skilled artisans.

In certain aspects, medical implants as described herein can comprise decontaminated adipose, dermis, or fascia, individually or in combination.

Medical implants as disclosed herein can be derived from allograft, autograft, xenograft, or synthetic

Described herein are methods of increasing growth factor binding. Methods of increasing growth factor binding as described herein can comprise providing a tissue, such as a bone composition; adding an acid to the solution, thereby exposing the bone composition to an acid to demineralize the bone composition; adding a buffer or base to raise the pH of the solution in the presence of one or more growth factors from a first level to a second level, wherein the second level is more basic than the first level, after at least partial demineralization of the bone. The bone composition can then be isolated, as can the mineral that has precipitated of solution (for example calcium), or both. In an embodiment, the bone extracellular matrix is removed prior to raising the pH in order to precipitate the calcium from the bone.

Buffers or bases as described herein can raise the pH of a composition or a solution from a first level to a second level, wherein the second level is more basic than the first level. In certain embodiments, buffers or bases as described herein can comprise sodium hydroxide, calcium hydroxide, potassium hydroxide, lithium hydroxide, rubidium hydroxide, strontium hydroxide, cesium hydroxide, barium hydroxide, water, sodium bicarbonate, tris, borate, carbonate, citrate, glycine, phosphate buffer, sodium chloride, media, or calcium salt. Bone compositions as described herein can comprise cortical bone, cancellous bone, or both. In certain aspects, the first pH level is an acidic pH level.

In certain aspects, medical implants as described herein are medical implants according to or created by any of the methods as described herein, individually or in combination.

Described herein is method of enriching bound growth factors in a medical implant, comprising: providing a scaffold; combining the scaffold with growth factors, peptides, or agents; raising the pH of the solution from a first level to a second level, wherein the second level is more basic than the first, by adding a base or buffer to the solution. After the pH is made more basic, the scaffold can be isolated. In certain aspects, the scaffold can be derived from allograft or xenograft tissue sources or created synthetically. In certain aspects, the scaffold can be comprised of comprised of collagen, hyaluronan, or other protein, amino acid, glycosaminoglycan (GAG), and/or biocompatible material (for example, calcium, salt, glycerol, or other storage agents or biological preservatives known in the art).

In some embodiments, target pH for the first level is under 1. In other embodiments, target pH for the first level is 1-1.5, 1.5-2, 2-2.5, 2.5-3, or 3-3.5. In some embodiments, the target pH for the second level is 7-7.5, 7.5-8, 8-8.5, 8.5-9, 9-9.5, 9.5-10, 10-10.5, 10.5-11

In other embodiments, the acid solution containing growth factors is not decanted prior to neutralization. The base or buffer is added directly to the solution containing growth factors. This is contrary to standard practice in the art, as solutions are typically decanted after treatment of tissue. In some embodiments, the tissue that the growth factors originated from is also included in the neutralization step.

In some embodiments, the acid solution used for the demineralization treatment or solution used for disinfection/decellularization, may be fractionated in application where the tissue is exposed to multiple acid or disinfection/decellularization solutions. These acids or disinfection/decellurization solutions may contain growth factors of interest. The solution used to recapture the bioactive elements and growth factors may be used in its entirety or select portions. The 1^{st} solution, 2^{nd} solution, 3^{rd} solution, 4^{th} solution, or 5^{th} solution may be utilized in part or in its entirety. Each solution may have unique attributes that may be desirable. For example, the solution from the 3^{rd} demineralization treatment of bone may contain highest growth factor content available for recapture and a low calcium content. In some embodiments, that 3^{rd} demineralization solution may be the most desirable for use. In other embodiments, the 1^{st} demineralization solution may be the most desirable for use.

In some embodiments, growth factors are derived from bone and are independent of the osseous tissue from which they are derived. Thus, growth factors, peptides, and mineralization proteins from cancellous bone, endosteum, or red bone marrow stroma may be combined with cortical bone. Similarly, growth factors derived from the periosteum may be combined with cancellous bone. Another example would be where growth factors derived from cells in the epidermis are combined with tissue from the dermis. Another example is when growth factors are derived from the synovial fluid are combined with cartilage tissue. Another example is where growth factors are derived from blood and combined with vasculature. In some embodiments, growth factors are derived from amniotic fluid and combined with amnion. In some embodiments, growth factors are derived from placental tissue and combined with placenta ECM. Similarly, proteins or mineral may be derived from cancellous bone and then be combined with cortical bone. One skilled in the art should understand there are many examples of growth factors being derived independent from the tissue from which is included in the implant.

In some embodiments, the rise in pH after applying a base or buffer may cause some minerals such as calcium or magnesium to precipitate out of solution. In these embodiments, the growth factors, peptides, or mineralization proteins may bind to the mineral as it becomes precipitate. The mineral may precipitate into a powdery particulate or, if the scaffold is present, it may form a coating on the scaffold. That coating may be uniform or non-homogenously deposited onto the scaffold. The coated surface of the scaffold may promote additional cell or protein binding. The growth factor, peptide, or mineralization protein that bound to the precipitate may result in a biphasic release profile once implanted into the recipient increasing its clinical efficacy.

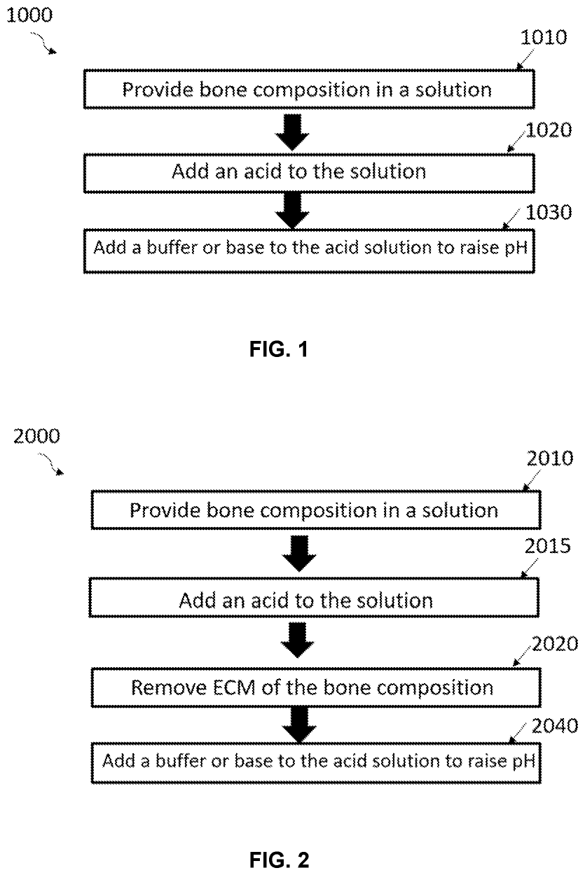

FIG. 1 is a flow chart illustrating an embodiment of the method 1000. According to the method 1000, a bone composition is provided in a solution 1010. The bone composition can comprise cortical bone, cancellous bone, or both. An acid can then be added to the solution 1020, followed by the addition of a base or buffer to the acid solution 1030 to raise the pH (make it more basic). Note that this is done without first decanting the acid solution. The buffer or base can comprise one or more of sodium hydroxide, calcium hydroxide, potassium hydroxide, lithium hydroxide, rubidium hydroxide, strontium hydroxide, cesium hydroxide, barium hydroxide, water, sodium bicarbonate, tris, borate, carbonate, citrate, glycine, phosphate buffer, sodium chloride, media, or calcium salt. The bone composition can then be isolated after the pH is raised. Acids used may be weak or strong. Acids may include, but not limited to hydrochloric or phosphoric, sulphuric, glycolic, acetic, paracetic, citric, ascorbic, and the like. The solution can further comprise one or more growth factors. The method can further comprise adding one or more growth factors at any stage of the method.

FIG. 2 is a flow chart illustrating an embodiment of the method 2000. According to the method 2000, a bone composition is provided in a solution 2010. The bone composition can comprise cortical bone, cancellous bone, or both. An acid can then be added to the solution 2015, and the extracellular matrix (ECM) is then removed from the bone composition 2020. Next is the addition of a base or buffer to the acid solution 2030 to raise the pH (make it more basic). Again, this is done without decanting the acid solution. The buffer or base can comprise one or more of sodium hydroxide, calcium hydroxide, potassium hydroxide, lithium hydroxide, rubidium hydroxide, strontium hydroxide, cesium hydroxide, barium hydroxide, water, sodium bicarbonate, tris, borate, carbonate, citrate, glycine, phosphate buffer, sodium chloride, media, or calcium salt. The bone composition can then be isolated after the pH is raised. Acids used may be weak or strong. Acids may include, but not limited to hydrochloric or phosphoric, sulphuric, glycolic, acetic, paracetic, citric, ascorbic, and the like. The solution can further comprise one or more growth factors. The method can further comprise adding one or more growth factors at any stage of the method. In some embodiments, the growth factors are derived from cells in the cancellous bone. For example, in some embodiments, the growth factors are released from cells within the cancellous bone during one or more processing steps and subsequently attach to the bone composition.

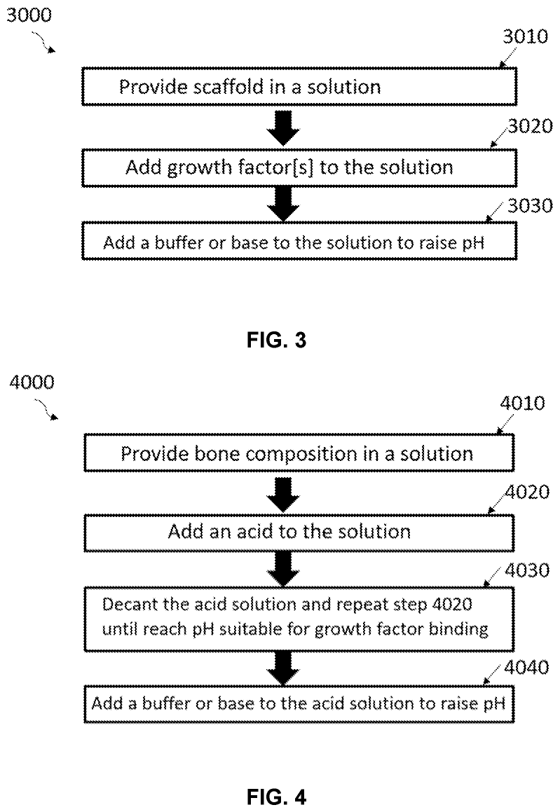

FIG. 3 is a flow chart illustrating an embodiment of the method 3000. According to the method 3000, a scaffold is provided in a solution 3010. The scaffold can be comprised of comprised of collagen, hyaluronan, or other protein, amino acid, GAG, and/or biocompatible material, and can be an allograft. Growth factors can then be added to the solution 3020, followed by the addition of a base or buffer to the acid solution 3030 to raise the pH (make it more basic). The scaffold can then be isolated after the pH is raised.

FIG. 4 is a flow chart illustrating an embodiment of the method 4000. According to the method 4000, a bone composition is provided in a solution 4010. The bone composition can comprise cortical bone, cancellous bone, or both. An acid can then be added to the solution 4020. The acid solution is then decanted but saved 4030, and step 4020 is repeated until a residual calcium level or target pH is obtained suitable for demineralizing the bone. At this point, a base or buffer is added to the acid solution 4040 (i.e., without decanting the acid solution) to raise the pH (make it more basic). One of the previously decanted acid solutions may be added to increase the growth factor content prior to base or buffer addition. The bone composition can then be isolated after the pH is raised. Acids used may be weak or strong. Acids may include, but not limited to hydrochloric or phosphoric, sulphuric, glycolic, acetic, paracetic, citric, ascorbic, and the like. The solution can further comprise one or more growth factors. The method can further comprise adding one or more growth factors at any stage of the method.

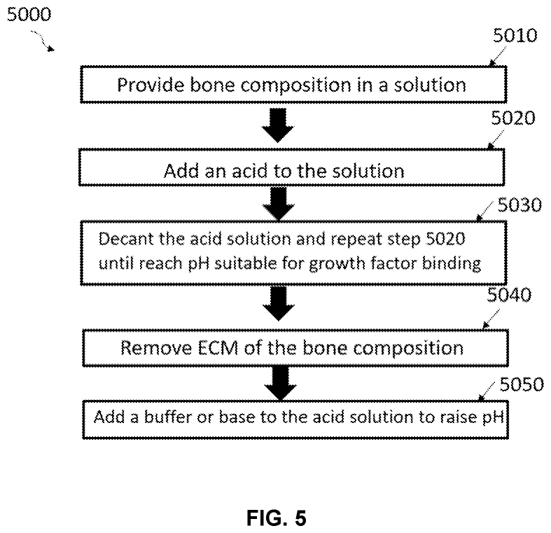

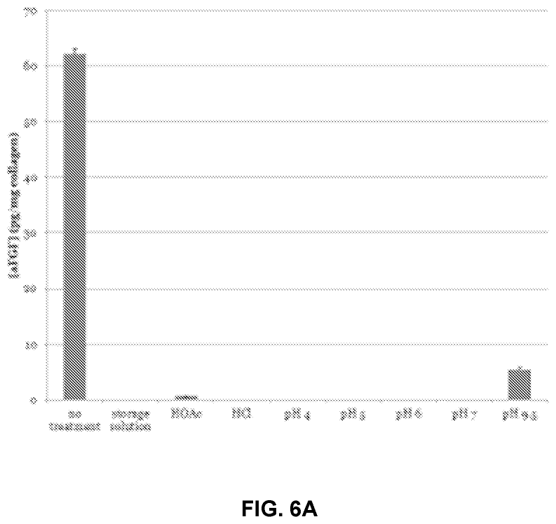

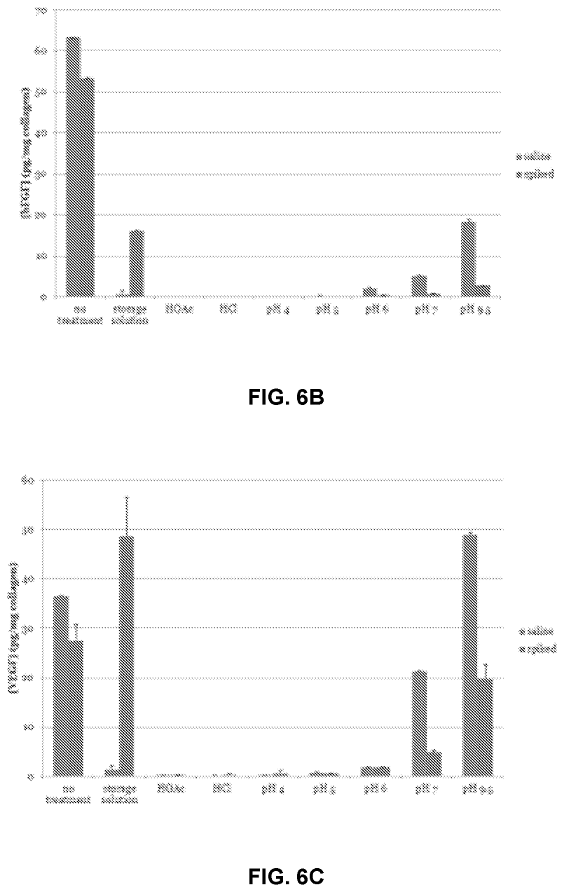

FIG. 5 is a flow chart illustrating an embodiment of the method 5000. According to the method 5000, a bone composition is provided in a solution 5010. The bone composition can comprise cortical bone, cancellous bone, or both. An acid can then be added to the solution 5020. The acid solution is then decanted 5040, and step 5030 is repeated until a target pH is obtained suitable for demineralizing the bone. The extracellular matrix (ECM) is then removed from the bone composition 5040. A base or buffer is then added to the acid solution 5050 (i.e., without decanting the acid solution) to raise the pH (make it more basic). The buffer or base can comprise one or more of sodium hydroxide, water, sodium bicarbonate, phosphate buffer, sodium chloride, or calcium salt. The bone composition can then be isolated after the pH is raised. Acids used may be weak or strong. Acids may include, but not limited to hydrochloric or phosphoric, sulphuric, glycolic, acetic, paracetic, citric, ascorbic, and the like. The solution can further comprise one or more growth factors. The method can further comprise adding one or more growth factors at any stage of the method.

Growth factors, for example, can comprise bone morphogenetic protein (BMP), vasoendothelial growth factor (VEGF), basic fibroblast growth factor (bFGF), acidic fibroblast growth factor (aFGF), transforming growth factor beta-1 (TGFB-1), platelet-derived growth factor (PDGF), insulin-like growth factor (IGF), hepatocyte growth factor (HGF), or any other growth factor or tissue growth stimulative agent or differentiation agent, individually or in combination, or other growth factors as described herein.

Medical implants as described herein can be made from autograft, allogeneic, or xenograft sources and may contain collagen, and growth factors/cytokines such as (but not limited to) BMP, PDGF, FGF, bFGF, aFGF, VEGF, hepatocyte growth factor (HGF), IGF, angiopoietin (ANG), ANG-2, fibronectin, TGFbl, etc. Components of implants as described herein can be mixed together or layered as an injectable or structured implant. Medical implants as described herein can be particulated implants, and in certain aspects can be crosslinked.

Medical implants described herein can be implanted surgically, injected, microneedled, and/or applied topically.

Medical implants described herein can be derived from follicular, dermis, fascia, amnion, amniotic fluid, placenta, umbilical cord, muscle, blood, bone, endosteum, bone stroma, bone marrow, or adipose tissue, their ECM, soluble proteins, or interacellular proteins.

Medical implants as described herein can be derived from a physiological solution containing cells such as blood, bone marrow, interstitial fluid, stromal vascular fraction, synovial fluid, interstitial fluid, amniotic fluid, and the like. The solution may also be derived from rinsing tissues mentioned with fluid, growth factors soluble in that fluid create a protein rich solution. Medical implants as described herein can be further purified using centrifugation, fluorescence, selective lysis, chromatography, precipitation, filtration, separation, and the like.

Additionally, medical implants as described herein can be refrigerated, frozen, or stored at ambient temperature. Medical implants as described herein can be dehydrated via lyophilization or supplied hydrated. Medical implants as described herein can be supplied in a syringe or a jar/bottle/vial.

Medical implants as described herein can be sterile filtered, tested per USP71, or terminally sterilized via irradiation (gamma, ebeam, uv, and the like).

Medical implants as described herein can be cleaned and disinfected using detergents, peroxides, antibiotics, water, and saline.

Medical implants as described herein can be cut into strips, sheets, fibers, or pieces. Medical implants can be ground or blended into fine particulate. Temperature control on cutting/grinding/blending may be used to help preserve growth factor content and prevent damage or denature proteins or other components.

Medical implant material (source tissue, final medical implants, or anything related to thereof or in between) may be screened/seived/filtered using syringes, needles, screens, seives, or filters. Tissue implant density may be controlled by filtration, dehydration, or centrifugation speeds (100-32000 rpm/g's).

Medical implants as described herein may have additives such as stabilizers (radioprotectants, lyoprotectants, or cryoprotectants, such as propylene glycol, glycerol, trehlose, sucrose, amino Acids, I-arginine, I-lysine, polysorbate, ascorbic acid, etc.). Additionally, medical implants can be mixed by shear stress prior to injection/implantation/application to improve flowability, decrease heterogenocity, and decrease particle size.

In certain embodiments, medical implants as described herein can comprise a backbone of one or more collagens.

Methods as described herein can utilize medical implants as described herein

Methods as described herein can deliver medical implants as described herein to a subject in need thereof. Medical implants can be delivered to the skin of a subject in need thereof.

Medical implants employed in methods as described herein can be compositions comprising growth factors. In certain aspects, growth factor compositions may also contain cells (such as stem cells, keratinocytes, adipocytes, adipose derived stem cells, bone marrow derived stem cells, perivascular cells, muscle cells, stromal vascular fraction, and the like). In addition, growth factor compositions as described herein can contain ascorbic acid, hemoglobin, oxygenation molecules, vasodilators, amino acids (such as arginine, lysine, methionine, cysteine, or the remaining 16 amino acids). In certain aspects, medical implants can comprise bone or cancellous bone. In certain embodiments, viable cells can be added to the medical implants after the medical implants are prepared.

As described herein, medical implants can comprise a bioactive intracellular component. A bioactive intracellular component can be a platelet-derived growth factor, a hepatocyte growth factor, an insulin growth factor, an angiopoietin, a fibronectin, a transforming growth factor, a nerve growth factor, a fibronectin, an integrin, a bone morphogenetic protein, an epidermal growth factor, an insulin-like growth factor, a fibroblast growth factor, vascular endothelial growth factor, osteoprotegerin, and osteopontin, and various combinations thereof.

As described, an effective amount of a tissue implant can be an amount of tissue implant that contains a bioactive intracellular component at a concentration of at least at least 1 pg/g. As described, an effective amount of a tissue implant can be an amount of tissue implant that contains a bioactive intracellular component at a concentration of about 0 pg/g to about 100 mg/g. An effective amount of a tissue implant can be an amount of tissue implant comprising a-fibroblast growth factor is present at a concentration of at least 1 pg/g. An effective amount of a tissue implant can be an amount of tissue implant comprising β-fibroblast growth factor is present at a concentration of at least 1 pg/g. An effective amount of a tissue implant can be an amount of tissue implant comprising vascular endothelial growth factor is present at a concentration of at least 1 pg/g. An effective amount of a tissue implant can be an amount of tissue implant comprising acidic fibroblast growth factor and is present at a concentration of at least 1 pg/g. An effective amount of medical implants as described herein administered to a subject in need thereof to treat soft-tissue defects can be an amount of tissue implant that contains a bioactive intracellular component at a concentration of at least at least 1 pg/mL. An effective amount of medical implants as described herein administered to a subject in need thereof to treat soft-tissue defects can be an amount of tissue implant that contains a bioactive intracellular component at a concentration of at least at least 10 pg/mL. An effective amount of medical implants as described herein administered to a subject in need thereof to treat soft-tissue defects can be an amount of tissue implant that contains a bioactive intracellular component at a concentration of at least at least 100 pg/mL. An effective amount of medical implants as described herein administered to a subject in need thereof to treat soft-tissue defects can be an amount of tissue implant that contains a bioactive intracellular component at a concentration of at least at least 1000 pg/mL. An effective amount of medical implants as described herein administered to a subject in need thereof to treat soft-tissue defects can be an amount of tissue implant that contains a bioactive intracellular component at a concentration of at least at least 10000 pg/mL. An effective amount of medical implants as described herein administered to a subject in need thereof to treat soft-tissue defects can be an amount of tissue implant that contains a bioactive intracellular component at a concentration of at least at least 100000 pg/mL.

An effective amount of medical implants as described herein administered to a subject in need thereof to treat soft-tissue defects can be an amount of tissue implant that comprises one or more of: aFGF in an amount of at least 100,000 pg/mL; FGF in an amount of at least 100,000 pg/mL; acidic fibroblast growth factor (aFGF) in an amount of at least 100,000 pg/mL; basic fibroblast growth factor (bFGF) in an amount of at least 100,000 pg/mL; epidermal growth factor (EGF) in an amount of at least 10,000 pg/mL; hepatocyte growth factor activator (HGFa) in an amount of at least 100,000 pg/mL; hepatocyte growth factor b (HGFb) in an amount of at least 100,000 pg/mL; insulin-like growth factor 1 (IGF-1) in an amount of at least 10,000 pg/mL; platelet derived growth factor BB in an amount of at least 10,000 pg/mL; transforming growth factor β1 (TGF-β1) in an amount of at least 10,000 pg/mL; and vascular endothelial growth factor (VEGF) in an amount of at least 5,000 pg/mL. In an embodiment, an amount effective comprises VEGF in an amount of about 5,000 pg/mL to about 1,000,000 pg/mL. In an embodiment, an amount effective comprises VEGF in an amount of about 66,000 pg/mL. Effective amounts of medical implants as described herein can be delivered to a subject in need thereof in a volume of about 0.01 cc to about 100 cc. Effective amounts of medical implants as described herein can be delivered to a subject in need thereof in a volume of about 0.01 cc to about 1 cc. Effective amounts of medical implants as described herein can be delivered to a subject in need thereof in a volume of about 1 cc to about 10 cc. Effective amounts of medical implants as described herein can be delivered to a subject in need thereof in a volume of about 10 cc to about 100 cc. Effective amounts of medical implants as described herein can be delivered to a subject in need thereof in a volume of about 10 cc. Effective amounts of medical implants as described herein can be delivered to a subject in need thereof in a volume of about 2 cc to about 9 cc. Effective amounts of medical implants as described herein can be delivered to a subject in need thereof in a volume of about 3 cc to about 8 cc. Effective amounts of medical implants as described herein can be delivered to a subject in need thereof in a volume of about 4 cc to about 7 cc. Effective amounts of medical implants as described herein can be delivered to a subject in need thereof in a volume of about 5 cc to about 6 cc. Effective amounts of medical implants as described herein can be delivered to a subject in need thereof in a volume of about 1 cc to about 20 cc. Effective amounts of medical implants as described herein can be delivered to a subject in need thereof in a volume of about 2 cc to about 19 cc. Effective amounts of medical implants as described herein can be delivered to a subject in need thereof in a volume of about 5 cc to about 15 cc.

Examples of soft tissues which can experience defects can include bone marrow, blood, adipose, skin, muscle, vasculature, cartilage, ligament, tendon, fascia, pericardium, nerve, and hair. These soft tissues may also include organs such as the pancreas, heart, kidney, liver, intestine, and stomach. In certain aspects, as used herein soft tissue can be any tissue for example, mesodermal, endodermal, and ectodermal tissues. Examples of these tissues include bone marrow, blood, adipose, skin, muscle, adipose, vasculature, cartilage, ligament, tendon, fascia, pericardium, nerve, and hair.

As such, the medical implants can be further sterilized to reduce the microorganism contamination to less than about 10⁻³ microorganisms. Typical sterilization methods include, but are not limited to, combinations of washing with or without pressurization, centrifugation with various chemicals such as alcohols and/or detergents and combining antibiotics with low-dose radiation. While these processing methods reduce the amount of microorganism contamination, they also can damage the medical implant and result in the loss of many intracellular proteins and molecules.

Other compositions, compounds, methods, devices, systems, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Soft tissues include any tissue or organ that is not bone, including, but not limited to adipose tissue, muscle, cartilage, tendons, and ligaments. In one embodiment, the harvested cells are adipose cells. The soft tissues can be autologous, allogeneic, xenogeneic, or syngeneic in origin. In order to minimize immunogenicity, the use of autologous cells is most advantageous. In other words, it is preferred if the harvested cells were obtained directly or indirectly (i.e., from an in vitro culture containing cells from the subject to receive the implant) from the subject that is to receive the soft tissue implant. In an embodiment, autologous adipose cells are harvested. In other embodiments, the tissue or cells are allogeneic.

Medical implants as described herein can comprise growth factors, particularly vascular endothelial growth factor (VEGF), basic fibroblast growth factor (bFGF), transforming growth factor beta 1 (TGFbl), acidic fibroblast growth factor (aFGF), insulin-like growth factor (IGF). Any given soft tissue protein and/or other bioactive factor can be present in the soluble soft tissue protein composition at a concentration of 0 pg/g to about 100 mg/g of isolated protein in the final product, dehydrated or otherwise provided.

In some embodiments, soluble soft tissue protein composition can include a stabilizer composition or stabilizer compounds. Suitable stabilization compounds can include, but are not limited to preservatives, antibiotics, antivirals, antifungals, pH stabilizers, osmostablizers, anti-inflammants, anti-neoplastics, chemotherapeutics, immunomodulators, chemoattractants, growth factors, anticoagulants, or combinations thereof. The stabilization solution can increase shelf life of the soft tissue soluble protein composition and/or reduce denaturation of proteins during dehydration, sterilization, and/or storage. In addition, other materials, such as nitrogen, can be used to help reduce free radical formation and denaturation during sterilization. In some embodiments, the stabilization solution per cc of final product can be about 1 mg sucrose, about 5 mg Glycine, about 3.7 mg I-Glutamic Acid, about 0.02 mg NaCl, and about 0.02 mg Polysorbate-80. In another embodiment, the stabilizer solution may contain glycerol, amino acids, polaxomers, carbomers, carbohydrates, polysaccharides, sugars, or salts.

In some embodiments, the final volume of a medical implant according to compositions and methods as described herein can be at least 1 cc, or 1 cc to about 100 cc, about 1 cc to about 50 cc, 1 cc to about 25 cc, about 1 cc to about 20 cc, about 1 cc to about 10 cc. The final soluble soft tissue protein product can be dehydrated or reconstituted to achieve a desired volume or particular protein concentration or composition.

Cells as described herein can be bone-marrow derived stem cells that are supported physiologically by a fibrous tissue called the stroma in the subject. There are two main types of stem cells in bone marrow: (1) hematopoietic stem cells and (2) bone marrow mesenchymal stem cells (bmMSCs). bmMSCs can differentiate into a variety of cell types including without limitation, fibroblasts, chondrocytes, osteocytes, myotubes, stromal cells, adipocytes, astrocytes, and dermal cells. In addition to bmMSCs, bone marrow stroma contains other types of cells including fibroblasts (reticular connective tissue) macrophages, adipocytes, osteoblasts, osteoclasts, red blood cells, white blood cells, leukocytes, granulocytes, platelets, and endothelial cells.

Proteins as described herein can be proteins derived from soluble bone marrow protein compositions that can contain proteins and/or other non-recombinant bioactive factors derived from bone marrow mesenchymal stem cells, fibroblasts, chondrocytes, osteocytes, red blood cells, white blood cells, leukocytes, granulocytes, platelets, and/or osteoclasts. The proteins can be intracellular proteins or membrane associated proteins. Such proteins include without limitation, bone morphogenetic proteins (BMPs) {e.g. BMP-1, BMP-2, BMP-3, BMP-4, BMP-5, BMP-7, BMP-8a, and BMP-9), transforming growth factors (TGF-βl, TGFB\hat{ }2, TGFB\hat{ }3), epidermal growth factor (EGF), hepatocyte growth factor (HGF), insulin-like growth factors (IGFs) (e.g. IGF-1), fibroblast growth factors (FGFs) (e.g. aFGF (acidic fibroblast growth factor) and bFGF (basic fibroblast growth factor)), vascular endothelial growth factor (VEGF), platelet derived growth factor-BB (PDGF-BB), osteoprotegerin (OPG), and osteopontin (OPN).

Additional examples of cells as described herein are adipose stem cells, apidocytes, mesenchymal stem cells, bone marrow stromal cells, progenitor cells, etc., that can remain viable in medical implants or in the tissue of the subject once the medical implants are implanted.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. Furthermore, one of skill in the art would recognize that the examples of the present disclosure presented below may be combined with one another.

### EMBODIMENTS

Embodiment 1. A method of increasing growth factor binding, comprising:
(a) providing a bone composition;
(b) adding an acid solution to the bone composition to lower the pH to an acidic level in an amount to at least partially demineralizing the bone composition; and
(c) adding a buffer or base to the acidic solution in an amount effective to precipitate minerals and promote binding of soluble growth factors in the acid solution to the bone composition, precipitated minerals, or a combination thereof.

Embodiment 2. The method of embodiment 1, further comprising decanting the acid solution from step (b) and repeating step (b) until a target pH or level of bone demineralization is reached prior to step (c).

Embodiment 3. The method of embodiment 2, wherein step (b) is repeated once prior to step (c).

Embodiment 4. The method of embodiment 2, wherein step (b) is repeated at least twice prior to step (c), wherein the method further comprises decanting the final acid solution after the target pH or bone demineralization is reached and adding the first decanted acid solution back to the bone composition prior to step (c).

Embodiment 5. The method of any one of embodiments 1 to 4, wherein pH for the acidic level is less than pH 1.0.

Embodiment 6. The method of any one of embodiments 1 to 4, wherein pH for the acidic level is from pH 1 to pH 3.5.

Embodiment 7. The method of any one of embodiments 1 to 6, wherein pH for the neutral or basic level is from pH 7 to pH 11.

Embodiment 8. The method of any one of embodiments 1 to 7, wherein the buffer or base comprises one or more of sodium hydroxide, calcium hydroxide, potassium hydroxide, lithium hydroxide, rubidium hydroxide, strontium hydroxide, cesium hydroxide, barium hydroxide, water, sodium bicarbonate, tris, borate, carbonate, citrate, glycine, phosphate buffer, sodium chloride, media, or calcium salt.

Embodiment 9. The method of any one of embodiments 1 to 8, further comprising isolating the precipitated minerals bound to growth factors.

Embodiment 10. The method of embodiment 9, wherein the precipitated minerals comprises calcium.

Embodiment 11. The method of any one of embodiments 1 to 10, wherein the bone composition comprises cortical bone, cancellous bone, or both.

Embodiment 12. A medical implant comprising a bone composition, mineral, or both prepared according to the methods of any one of embodiments 1 to 11.

Embodiment 13. A method of enriching bound growth factors in a medical implant, comprising:
(a) providing a scaffold in solution;
(b) combining the scaffold in solution with growth factors or agents; and
(c) raising the pH of the solution from a first level to a second level,
wherein the second level is more basic than the first, by adding a base or buffer to the solution.

Embodiment 14. The method of embodiment 13, wherein the scaffold is derived from allograft or xenograft tissue sources or created synthetically.

Embodiment 15. The method of embodiment 13 or 14, wherein the scaffold is comprised of comprised of collagen, hyaluronan, or other protein, amino acid, GAG, and/or biocompatible material.

Embodiment 16. The method of any one of embodiments 1 to 15, wherein the growth factors or agents comprise bone morphogenetic protein (BMP), vasoendothelial growth factor (VEGF), basic fibroblast growth factor (bFGF), acidic fibroblast growth factor (aFGF), transforming growth factor beta-1 (TGFB-1), platelet-derived growth factor (PDGF), insulin-like growth factor (IGF), hepatocyte growth factor (HGF), or any other growth factor or tissue growth stimulative agent or differentiation agent, individually or in combination.

Embodiment 17. The method of any one of embodiments 1 to 16, wherein the first level is an acidic pH.

Embodiment 18. The method of any one of embodiments 1 to 7, wherein the buffer or base comprises one or more of sodium hydroxide, calcium hydroxide, potassium hydroxide, lithium hydroxide, rubidium hydroxide, strontium hydroxide, cesium hydroxide, barium hydroxide, water, sodium bicarbonate, tris, borate, carbonate, citrate, glycine, phosphate buffer, sodium chloride, media, or calcium salt.

Embodiment 19. A medical implant produced by the method of any one of embodiments 13 to 18.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Enrichment and Increased Binding of Growth Factors

Described herein are medical implants that can be implanted/injected into a subject, or applied onto a subject in a location where growth factors/agents can be exposed to biocompatible scaffolding material where the combination of scaffold/growth factor solution has an acidic pH.

The scaffold may be acidic and/or the growth factors can be in an acidic medium. The binding of those growth factors onto the scaffold can be increased by bringing the pH of the growth factors/scaffold combination to a higher pH. The pH change can approach about a neutral pH (>=4) or go into a basic pH. The pH can be elevated using a buffer or a base. Growth factors may consist of proteins, cytokines, peptides, amino acids, etc.

In some embodiments, target pH for the first level is under 1. In other embodiments, target pH for the first level is 1-1.5, 1.5-2, 2-2.5, 2.5-3, or 3-3.5. In some embodiments, the target pH for the second level is 7-7.5, 7.5-8, 8-8.5, 8.5-9, 9-9.5, 9.5-10, 10-10.5, or 10.5-11.

Growth factors/agents according to the present example may be BMP, HGF, VEGF, bFGF, aFGF, TGFB-1, PDGF, IGF, or any other growth factor or tissue growth simulative agent or differentiation agent.

Scaffolds may be derived from allograft or xenograft tissue sources or created synthetically. The scaffolds may be at least partially comprised of collagen, hyaluronan, or other protein, amino acid, GAG, and/or biocompatible material

Acids used may be weak or strong. Acids may include, but not limited to hydrochloric or phosphoric, sulphuric, glycolic, acetic, paracetic, citric, ascorbic, and the like.

Bases or buffers can be sodium hydroxide (NaOH), water, sodium bicarbonate, phosphate buffer, sodium chloride (NaCl), Calcium salt, or any other material that will raise pH of an acidic solution.

In an embodiment of the present example, bone can be combined with growth factors/agents with an acidic pH. The pH of the bone/growth agents can then be raised by the addition of sodium bicarbonate. The resulting bone is growth factor enriched and growth factors/agents are bound to the bone at a level higher than before treatment with the sodium bicarbonate.

In another embodiment, growth factors bind to the calcium, other mineral, or other insoluble fraction of the acidic solution precipitating out of solution as the pH is raised.

In some embodiments of the present example, allograft tissue can be combined with growth factors/agents with an acidic pH. The pH of the growth agents can then be raised by the addition of sodium bicarbonate or NaOH. The resulting tissue is growth factor enriched and growth factors/agents are bound to the adipose at a higher level than before sodium bicarbonate. Neutralization of the pH to 5 resulted in lower growth factor content than neutralization to pH 6. Neutralization of the pH to 6 resulted in lower growth factor content than neutralization to pH 7. Neutralization of the pH to 7.0 resulted in lower growth factor content than neutralization to pH less than about 7.5.

Neutralization of the pH to 7.5 resulted in lower growth factor content than neutralization to pH less than about 8.0. Neutralization of the pH to 8.0 resulted in lower growth factor content than neutralization to pH less than about 8.5. Neutralization of the pH to 8.5 resulted in lower growth factor content than neutralization to pH less than about 9.0. Neutralization of the pH to 9 resulted in lower growth factor content than neutralization to pH less than about 9.5. Neutralization of the pH to 9.5 resulted in lower growth factor content than neutralization to pH less than about 10. Neutralization of the pH to 10 resulted in lower growth factor content than neutralization to pH less than about 10.5. Neutralization of the pH to 10.5 resulted in lower growth factor content than neutralization to pH less than about 11. Neutralization of the pH to pH higher than 11 resulted in higher growth factor content than neutralization to pH less than about 11.

In an embodiment of the present example, a scaffold containing collagen can be combined with growth factors/agents with an acidic pH. The collagen/growth agents are then raised in pH by the addition of sodium bicarbonate. The resulting collagen is growth factor enriched and growth factors/agents are bound to the collagen at a higher level.

In an embodiment of the present example, mineralized cortical or cancellous bone (sourced from allograft or xenograft tissue sources or created synthetically) is exposed to up to 1 M HCL or other acid (0.5-0.6N for example), and then after at least partial demineralization, the solution pH is raised by adding a buffer or base to above pH 8. The growth factors and growth agents that were bound to the mineral (and loosely bound to collagen or lipid) are released into the solution and raising the pH causes those growth factors/agents to bind to the extracellular matrix of the bone. Also, mineral (such as calcium) may precipitate out of solution and have attached growth factors/agents.

### Example 2: Medical Implant with Elevated VEGF Concentration

Disclosed according to the present example is a medical implant derived from blood that has an elevated VEGF concentration by lysing and harvesting VEGF from the intracellular space of red blood cells. The blood tissue is exposed to acid and then the solution pH is raised and filtered/centrifuged allowing for implantation or injection. The implant may be used in combination with platelets from that same donor. Implant may be frozen, freeze-dried, allogeneic, autologous, or xenographic.

### Example 3: Quantification of Translocated Protein and Growth Factors Derived from Demineralized Cortical Fibers

The purpose of this protocol is to quantifiably assess protein and select growth factor (GF) translocation post cortical fiber demineralization neutralization via Bicinchoninic Acid Assay (BCA) and Quantibody ELISA Array fabricated by RayBiotech (Peachtree Corners, GA).

Upon demineralization of cortical bone with hydrochloric acid, it has been shown that some proteins can be stripped from the tissue and become solubilized in the resulting solution (Pietrzak et al. 2009) which is a mixture of residual acid and salts, solubilized proteins, and solubilized mineral. Upon neutralization, the mineral component can be precipitated. This study will demonstrate the fate of various proteins of interest from the demineralization process and whether they end up still bound to the tissue, bound to mineral phase, or present in the remaining solution (henceforth referred to as the “protein rich solution”).

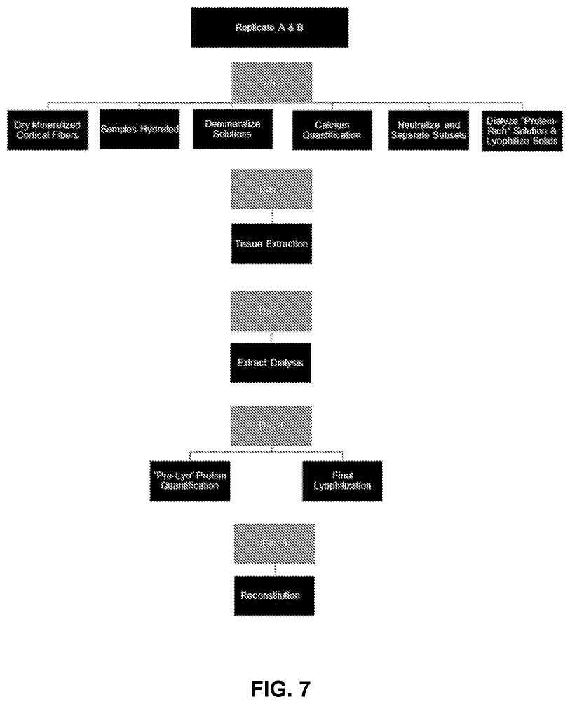

FIG. 7 illustrates the study protocol.

Overall, total protein concentration is highest in Mineral Particle extract, followed by tissue extract, then Protein-Rich Solution one. This is summarized in Table 1. Further analysis via protein array showed that greater than 50% of the total growth factor content for aFGF, BMP-9, HGF, and TGFb-1 were recovered in the first extract solution while other growth factors like bFGF were more present in the second extract solution. Other growth factors such as OPG and VEGF-A were in considerable abundance in the protein rich solution.

TABLE 1

| | Total protein (μg) - | Standard |
|---|---|---|
| | reconstituted product | Dev. |
| Protein-Rich Solution 1 | 10,287.19 | 259.28 |
| Mineral Particle (Extract) 1 | 103,131.12 | 24,841.74 |
| Mineral Particle (Extract) 2 | 2,341.02 | 441.21 |
| Tissue (Extract) | 81,559.26 | 3,538.84 |

Unless defined otherwise, all technical and scientific terms used have the same meaning as commonly understood by one of ordinary skill in the art to which this disclosure belongs. Although any methods and materials similar or equivalent to those described can also be used in the practice or testing of the present disclosure, the preferred methods and materials are now described.

Embodiments of the present disclosure will employ, unless otherwise indicated, techniques of separating, testing, and constructing materials, which are within the skill of the art. Such techniques are explained fully in the literature.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

What is claimed is:

1. A method of increasing growth factor binding, comprising:
(a) providing a bone composition;
(b) adding an acid solution to the bone composition to lower the pH to an acidic level in an amount to at least partially demineralizing the bone composition; and
(c) adding a buffer or base to the acidic solution in an amount effective to precipitate minerals and promote binding of soluble growth factors in the acid solution to the bone composition, precipitated minerals, or a combination thereof.

2. The method of claim 1, further comprising decanting the acid solution from step (b) and repeating step (b) until a target pH or bone demineralization is reached prior to step (c).

3. The method of claim 2, wherein step (b) is repeated once prior to step (c).

4. The method of claim 2, wherein step (b) is repeated at least twice prior to step (c), wherein step (b) further comprises decanting the final acid solution after the target pH or bone demineralization is reached and adding the first decanted acid solution back to the bone composition prior to step (c).

5. The method of claim 1, wherein pH for the acidic level is less than pH 1.0.

6. The method of claim 1, wherein pH for the acidic level is from pH 1 to pH 3.5.

7. The method of claim 1, wherein pH for the neutral or basic level is from pH 7 to pH 11.

8. The method of claim 1, wherein the buffer or base comprises one or more of sodium hydroxide, calcium hydroxide, potassium hydroxide, lithium hydroxide, rubidium hydroxide, strontium hydroxide, cesium hydroxide, barium hydroxide, water, sodium bicarbonate, tris, borate, carbonate, citrate, glycine, phosphate buffer, sodium chloride, media, or calcium salt.

9. The method of claim 1, further comprising isolating the precipitated minerals bound to growth factors.

10. The method of claim 9, wherein the precipitated minerals comprises calcium.

11. The method of claim 1, wherein the bone composition comprises cortical bone, cancellous bone, or both.

12. A medical implant comprising a bone composition, mineral, or both prepared according to the methods of claim 1.

13. A method of enriching bound growth factors in a medical implant, comprising:
(a) providing a scaffold;
(b) combining the scaffold in solution with growth factors or agents;
(c) raising the pH of the solution from a first level to a second level,
wherein the second level is more basic than the first, by adding a base or buffer to the solution.

14. The method of claim 13, wherein the scaffold is derived from allograft or xenograft tissue sources or created synthetically.

15. The method of claim 13, wherein the scaffold is comprised of comprised of mineral, collagen, hyaluronan, or other protein, amino acid, GAG, and/or biocompatible material.

16. The method of claim 13, wherein the growth factors or agents comprise bone morphogenetic protein (BMP), vasoendothelial growth factor (VEGF), basic fibroblast growth factor (bFGF), acidic fibroblast growth factor (aFGF), transforming growth factor beta-1 (TGFB-1), platelet-derived growth factor (PDGF), insulin-like growth factor (IGF), hepatocyte growth factor (HGF), or any other growth factor or tissue growth stimulative agent or differentiation agent, individually or in combination.

17. The method of claim 13, wherein the first level is an acidic pH.

18. The method of claim 13, wherein the buffer or base comprises one or more of sodium hydroxide, calcium hydroxide, potassium hydroxide, lithium hydroxide, rubidium hydroxide, strontium hydroxide, cesium hydroxide, barium hydroxide, water, sodium bicarbonate, tris, borate, carbonate, citrate, glycine, phosphate buffer, sodium chloride, media, or calcium salt.

19. A medical implant produced by the method of claim 13.
